(19) United States
(12) Patent Application Publication
(10) Pub. No.: US 2012/0142628 A1
(43) Pub. Date: Jun. 7, 2012

(54) METHODS FOR TREATING CREPITUS

(75) Inventor: Dinusha N. Lalwani, Corona, CA (US)

(73) Assignee: ALLERGAN, INC., Irvine, CA (US)

(21) Appl. No.: 13/314,075

(22) Filed: Dec. 7, 2011

Related U.S. Application Data
(60) Provisional application No. 61/420,651, filed on Dec. 7, 2010.

(51) Int. Cl. A61K 31/728; A61P 19/02

(52) U.S. Cl. 514/54

(57) ABSTRACT

Methods for treating and/or preventing crepitus and other joint problems using hyaluronic acid based formulations are provided.

The present application claims priority to U.S. Provisional Patent Application No. 61/420,651, filed Dec. 7, 2010, the entire disclosure of which is incorporated herein by this specific reference.

The present invention generally relates to methods for treating joint disorders and/or diseases, and more specifically relates to methods for treating symptoms of crepitus, pain and/or prevention of joint space narrowing, for example, such joint disorders associated with osteoarthritis (OA), injury or joint irregularities, using hyaluronic acid based formulations.

Osteoarthritis (OA), the most common form of arthritis, is a chronic disease characterized by the slow degradation of cartilage pain and increasing disability. Between 2% and 10% of the adult U.S. population is affected by OA of the knee and a small but significant proportion develops severe joint damage leading to surgery.

There are several signs and symptoms associated with osteoarthritis and a notable common presenting sign associated with the early stages of osteoarthritis development is a condition termed “crepitus”.

Crepitus is a medical term to describe the audible grating, crackling and/or popping sounds and palpable grating, popping, clicking or cracking sensations experienced under the skin and joints. The cause of crepitus is recognized as normally due to a deterioration or absence of cartilage and meniscus in the joint structure, or can be due to cavitation of gas in the joints or other joint irregularities. Crepitus is commonly present without pain and may or may not signify the presence of arthritis or joint injury.

Injectable, hyaluronic acid-based formulations indicated for treating OA are commercially available. However, these prior art formulations typically have a low hyaluronic acid concentration, often a HA concentration of 10 mg/ml or less, and have a low viscosity to provide for lubrication of joint surfaces.

### SUMMARY

Accordingly, the present invention provides methods of treatment using certain hyaluronic acid-based formulations to treat or prevent joint pathologies, for example, joint space narrowing or crepitus. More specifically, hyaluronic acid-based formulations useful in accordance with the invention include high concentration hyaluronic acid based gels, including crosslinked highly cohesive HA based gels, such as crosslinked hyaluronic acid-based gels indicated for treating wrinkles and folds in skin, for example, but not limited to dermal fillers, for example, dermal fillers marketed under the tradename JUVÉDERM®.

In another aspect of the invention, methods are provided for correcting the patellofemoral joint space between uneven articular joint surfaces by administering in a region of a joint, injections of cross linked hyaluronic acid, for example, JUVÉDERM®, in order to increase synovial fluid volume and to prevent further joint space narrowing.

In yet other aspects of the invention, these hyaluronic acid based formulations are utilized injected into a joint space for palliative treatment associated with osteoarthritis.

In one embodiment, methods are provided for treating crepitus using a hyaluronic acid-based formulation comprising cross-linked hyaluronic acid formulated to a concentration of at least about 20 mg/ml up to about 30 mg/ml, for example, about 24 mg/ml (molecular weight (MW) of between about 1.5 MDa to about 2.5 MDa), or more, which is suspended in a physiological buffer and indicated for correction of moderate to severe facial wrinkles and folds.

With existing damage to articular cartilage, the methods of the present invention may be used to restore movement and to reduce crepitus, for example, patellofemoral crepitus.

In one aspect, a method for treating or preventing crepitus in a patient is provided wherein the method comprises introducing, near or into a joint affected by crepitus, an effective amount of a hyaluronic acid-based dermal filler composition. The joint may be any joint affected by crepitus. The crepitus may be present in the affected joint with or without pain or joint damage.

In one aspect, the composition is injected into a joint space of the affected joint. In an affected knee joint, the joint space may be the patellofemoral joint space. In an affected shoulder joint, the joint space may be the glenohumeral joint space.

In another aspect, a method for treating or preventing crepitus in a joint is provided, the method comprising introducing, into a joint region of a joint prone to crepitus, an effective amount of a crosslinked hyaluronic acid based composition having a hyaluronic acid concentration of between about 20 mg/ml and 30 mg/ml.

### DETAILED DESCRIPTION

Methods of managing joint health are provided, for example, methods which are useful as a treatment or preventive measure targeting such joint problems as joint space narrowing and crepitus, presenting with or without pain, using known hyaluronic acid-based compositions. The joint that may be treated with the present methods includes, but is not limited to, any joint that may benefit from the methods, for example, such joints that are commonly affected by problems such as crepitus, pain and joint space narrowing. Such joints include, but not limited to, a patellofemoral joint, glenohumeral joint, or temporomandibular joint. The compositions used in the methods of the invention may be, for example, those commercially available compositions presently manufactured and indicated for use as dermal fillers, for example, those marketed under the tradename JUVÉDERM®, including JUVÉDERM® Ultra, JUVÉDERM® Ultra Plus, JUVÉDERM® Ultra XC, and JUVÉDERM® Ultra Plus XC, available from Allergan, Inc., or other compositions that are compositionally similar or identical to such dermal filler compositions.

In one aspect of the invention, dermal filler compositions are used to prevent or ameliorate joint space narrowing and/or crepitus, by injection of an effective amount of the composition into a joint region, for example, into a joint space of the affected joint.

For example, in one embodiment, a dermal filler composition, for example, a commercially available hyaluronic acid based, dermal filler composition, or a formulation or composition compositionally identical thereto, is introduced into a joint space that is subject to possible joint space narrowing, or a joint that may be experiencing crepitus upon movement. The composition expands the joint space a sufficient amount to provide relief from crepitus or pain associated with a narrow joint space. In some embodiments, no active agents are introduced in conjunction with the composition. In other embodiments, no active agents other than lidocaine or its equivalent is introduced in conjunction with the composition.

In accordance with one aspect, prophylactic injections of the compositions are administered to patients who do not have osteoarthritis in the joint, but have a predisposition to osteoarthritis either due to positive family history, injury or occupational/professional concerns that relate to significant wear and tear to the joint cavity.

In some embodiments of the invention, the compositions injected near or into the joint space to treat or prevent a joint disorder or disease are absent of active ingredients. In other embodiments, the compositions include at least one active ingredient, for example, an anesthetic agent such as, but not limited to, lidocaine. In many embodiments of the present invention, the compositions are chemically/structurally identical to commercially available dermal filler compositions, such as those dermal filler compositions manufactured and sold by Allergan, Inc, for example, the dermal filler marketed under the name Juvederm® Ultra.

In one embodiment, the joint is a knee joint, for example a patellofemoral joint. The method may include, for example, injecting about 0.5 cc to about 2.0 cc, for example, about 0.8 cc of the composition into the subject patellofemoral joint at 1 injection for about 3 consecutive weeks for the initial treatment. The treatment may be continued throughout the lifetime of the patient, for example, at intervals of every four to six months, to prevent the occurrence of joint problems throughout the life of the patient. The somewhat continuous presence of the composition in the joint space reduces or eliminates crepitus, and/or joint space narrowing, and may prevent, or at least reduce the possibility of arthritic joint degradation, or further degradation of the joint by restoring joint fluid to cushion, protect and lubricate the joint.

In some embodiments, the methods comprise introducing, into or near a joint space, an effective amount of a crosslinked hyaluronic acid based composition having a hyaluronic acid concentration of between about 10 mg/ml and 40 mg/ml, for example, between about 20 mg/ml and about 30 mg/ml, for example, greater than 10 mg/ml, greater than about 15 mg/ml, greater than about 20 mg/ml, for example, about 24 mg/ml. In a more specific embodiment, the methods comprise introducing into a joint space, an effective amount of a crosslinked hyaluronic acid based composition comprising a crosslinked hyaluronic acid having a molecular weight of about 1.5 million Daltons to about 2.5 million Daltons and a hyaluronic acid concentration of at least 20 mg/ml, for example, about 24 mg/ml.

In one embodiment, a method for treating or preventing crepitus in a knee joint is provided, the method comprising the steps of introducing, into a patellofemoral joint space, an effective amount of a crosslinked hyaluronic acid based composition including lidocaine, the composition having a hyaluronic acid concentration of between about 20 mg/ml to about 30 mg/ml, the crepitus in the knee joint being present without pain in the knee joint. The composition is a commercial, crosslinked hyaluronic acid-based dermal filler composition.

In another embodiment, a method for treating or preventing crepitus in a shoulder joint is provided, the method comprising the steps of introducing, into a glenohumeral joint space, an effective amount of a crosslinked hyaluronic acid based composition including lidocaine, the composition having a hyaluronic acid concentration of between about 20 mg/ml to about 30 mg/ml, the crepitus in the shoulder joint being present without pain in the shoulder joint. The composition is a commercial, crosslinked hyaluronic acid-based dermal filler composition.

In yet another embodiment, a method for treating or preventing crepitus in a mandibular joint is provided, the method comprising the steps of introducing, into a temporomandibular joint space, an effective amount of a crosslinked hyaluronic acid based composition including lidocaine, the composition having a hyaluronic acid concentration of between about 20 mg/ml to about 30 mg/ml, the crepitus in the mandibular joint being present without pain in the jaw. The composition is a commercial, crosslinked hyaluronic acid-based dermal filler composition.

### EXAMPLE 1

### Treatment of Crepitus in the Injured Knee Joint

30 year old female patient presents with joint stiffness and pain in the right knee following a fall from a ladder a few weeks ago. Patient states that the pain gets worse with walking long distances and improves with rest. Patient has a long family history for osteoarthritis and would like treatment to prevent progression of disease and well as treating the sound that the knee produces when the patient walks. Physical examination shows decreased range of passive movement and palpable crepitus upon flexion and extension >90 degrees. Mild inflammation around patellar region noted. No other contributing factors noted on physical examination. Radiograph imaging reveals presence of osteophytes (spurs) at joint margins. The physician administers 3 bilateral treatments of a commercial, crosslinked hyaluronic acid-based dermal filler composition, such as Juvederm XC (initial treatment and 2 additional treatments) of 1.6 cc (0.8 cc×2) approximately 1 week apart using aseptic techniques with a 27 gauge needle. During routine follow up visit two weeks after the final treatment, the patient states that the right knee no longer makes a creaking sound when she walks. Physician concludes that crepitus has resolved with increase in range of motion which is consistent with radiographic studies, patient survey information and physical findings.

### EXAMPLE 2

### Treatment of Crepitus in the Knee Joint, Crepitus Exhibiting without Pain

59 year old soccer player presents with crepitus in the right knee. His complaint is of “a funny sound he hears when he runs” during his weekend soccer games. He reports that he does not feel any pain in the knee, but that the creaking sound is embarrassing and distracting, and he would like it corrected.

Examination reveals patellofemoral crepitus upon passive flexion of his right knee. An X-ray of his knee reveals some loss of joint space. The physician administers 3 injection treatments of 0.8 cc of Juvederm XC, an injectable crosslinked hyaluronic acid-based gel having a hyaluronic acid concentration of 24 mg/mL and 0.3% w/w lidocaine in a physiologic buffer, each injection treatment at approximately 1 week apart using aseptic techniques with a 27 gauge needle. Patient returns for follow up visit and reports and increase in range of motion and ability to run longer distances without any more “funny” sounds from his knee. Patient returns for treatment about every six months for as long as he continues to play weekend soccer.

### EXAMPLE 3

### Treatment of Patellofemoral Crepitus in Runner's Knee

A 38 year old woman presents with pain around her knee cap and popping and grinding sensation around her knee. She had been diagnosed with “runner's knee” and has been taking ibuprofen as prescribed by her doctor, in addition to wearing arch support and elevating her knee when she has pain. She would like to know how she can alleviate the grinding sound in her knee so that she can continue to run and reduce the need for ibuprofen. Physical examination reveals crepitus on palpation of patellofemoral area bilaterally. No tenderness or redness is noted. Patient has normal range of movement on both legs and no other significant findings on physical exam. Physician administers 3 bilateral treatments (initial treatment and 2 additional treatments) of 1.6 cc (0.8 cc×2) approximately 1 week apart using aseptic techniques with a 27 gauge needle. Patient returns for follow up visit and states that the grinding sound in her knee has gone away and there is less pain following running.

### EXAMPLE 4

### Treatment for Crepitus and Joint Space Narrowing

45 year old female presents to office with difficulty walking in both legs. Patient has a past medical history significant for hypertension, diabetes and osteoarthritis. Patient complains that she feels like she has “rice crispies” in her knees upon exertion. Patient is reluctant to take pharmacotherapies for treatment because she wants to “minimize” dependence of medication. Patient wants to know what she can do to prevent her condition from getting worse. Palpable crepitus observed in knees bilaterally with limited range of movement on extension and passive flexion. Radiographic tests reveal moderate narrowing of the medial, lateral and patellofemoral joint compartments consistent with osteoarthritis of the right and left knee.

The physician treats the patient with 3 bilateral treatments (initial treatment and 2 additional treatments) of 1.0 cc (0.5 cc×2) of Juvederm Ultra approximately 1 week apart using aseptic techniques with a 27 gauge needle. Routine follow up visits every 2 weeks. Patient returns for follow up visit and radiographic tests/MRI are consistent with increased patellofemoral joint space and increased synovial fluid volume/hyaluronate confirmed by arthrocentesis.

### EXAMPLE 5

### Treatment for Crepitus in a Shoulder

42 yr male presents to physician with concerns about a left shoulder injury sustained during fall from a horse two years prior. Patient complains of audible grinding and popping sounds when rotating his shoulder, but says that he does not feel pain in the shoulder. Patient exercises regularly and is not taking any current medications except a multivitamin daily.

Physical exam of musculoskeletal system reveals significant palpable crepitus in left glenohumeral joint on rotation of the shoulder. Radiographic tests reveal moderate narrowing of both the left and right glenohumeral joint compartments. The patient is treated with 4 bilateral treatments of a highly cohesive crosslinked hyaluronic acid based formulation (20 mg/ml HA) without active agents (initial treatment and 3 additional treatments) of 1.6 cc (0.8 cc×2) approximately 2 weeks apart using aseptic techniques with a 25 gauge needle into the glenohumeral joint compartments. Routine follow up visits every 2 weeks. Patient returns for follow up visit and radiographic tests/MRI are consistent with increased glenohumeral joint space and increased synovial fluid volume/hyaluronate confirmed by arthrocentesis and physical examination. Patient returns for treatments about once a year thereafter.

What is claimed is:

1. A method for treating or preventing crepitus in a patient, the method comprising:
introducing, into a joint region of a joint affected by crepitus, an effective amount of a hyaluronic acid-based dermal filler composition.

2. The method of claim 1 wherein the joint is a knee joint.

3. The method of claim 2 wherein the joint is a patellofemoral joint.

4. The method of claim 1 wherein the joint is a shoulder joint.

5. The method of claim 4 wherein the joint is a glenohumeral joint.

6. The method of claim 1 wherein the crepitus is present in the patient without pain in the affected joint.

7. The method of claim 1 wherein the step of introducing comprises injecting the composition into a joint space of the affected joint.

8. The method of claim 1 wherein the effective amount of the composition is between about 0.5 cc and about 2 cc.

9. The method of claim 1 wherein the composition is a crosslinked hyaluronic acid-based composition.

10. The method of claim 1 wherein the composition is a crosslinked hyaluronic acid based composition having a hyaluronic acid concentration in a range of between about 20 mg/ml to about 30 mg/ml.

11. The method of claim 1 wherein the composition is a crosslinked hyaluronic acid based composition having a hyaluronic acid concentration of about 24 mg/ml.

12. A method for treating or preventing crepitus in a joint comprising:
introducing, into a joint region of a joint prone to crepitus, an effective amount of a crosslinked hyaluronic acid based composition having a hyaluronic acid concentration of between about 20 mg/ml and 30 mg/ml.

13. The method of claim 12 wherein the hyaluronic acid concentration is about 24 mg/ml.

14. The method of claim 12 wherein the joint is a knee joint.

15. The method of claim 14 wherein the joint is a patellofemoral joint.

16. The method of claim 12 wherein the joint is a shoulder joint.

17. The method of claim 16 wherein the joint is a glenohumeral joint.

18. The method of claim 12 wherein the composition is a commercial hyaluronic acid based dermal filler.

19. A method for treating or preventing crepitus in a knee joint comprising:
introducing, into a patellofemoral joint space between about 0.5 cc to about 2.0 cc, an effective amount of a crosslinked hyaluronic acid based composition including lidocaine, the composition having a hyaluronic acid concentration of between about 20 mg/ml to about 30 mg/ml, the crepitus in the knee joint being present without pain.

20. The method of claim 19 wherein the composition is a commercial, crosslinked hyaluronic acid-based dermal filler composition.
